(19) United States
(12) Patent Application Publication
(10) Pub. No.: US 2020/0312432 A1
(43) Pub. Date: Oct. 1, 2020

(54) COMPUTER ARCHITECTURE FOR LABELING DOCUMENTS

(71) Applicant: 3M INNOVATIVE PROPERTIES COMPANY, SAINT PAUL, MN (US)

(72) Inventors: GUOLI WANG, NORTH POTOMAC, MD (US); HAN-CHIN SHING, GREENBELT, MD (US)

(73) Assignee: 3M INNOVATIVE PROPERTIES COMPANY, SAINT PAUL, MN (US)

(21) Appl. No.: 16/829,221

(22) Filed: Mar. 25, 2020

Related U.S. Application Data
(60) Provisional application No. 62/826,128, filed on Mar. 29, 2019.

(51) Int. Cl. G16H 15/00; G06F 16/93; G06K 9/62; G06N 3/08; G06N 3/04; G06N 20/20; G06F 17/18

(52) U.S. Cl. CPC G16H 15/00; G06F 16/93; G06K 9/6256; G06F 17/18; G06N 3/0454; G06N 20/20; G06N 3/084

(57) ABSTRACT

A computer architecture for labeling documents is disclosed. According to some aspects, a computer accesses a collection of documents corresponding to a medical encounter and a labeling for the collection, wherein the labeling comprises one or more labels representing medical annotations assigned to the medical encounter. The computer computes, using a Hierarchical Attention Network (HAN), for each of a plurality of document-label pairs, a probability that a document of the document-label pair corresponds to a label of the document-label pair based on one or more features of text in the document, wherein each document-label pair comprises a document from the collection of documents and a label from the labeling. The computer provides an output representing the computed probabilities.

### CROSS-REFERENCE TO RELATED APPLICATION

This application claims priority from U.S. Provisional Application Ser. No. 62/826,128, filed Mar. 29, 2019, the disclosure of which is incorporated by reference in its entirety herein.

### TECHNICAL FIELD

Embodiments pertain to computer architecture. Some embodiments relate to machine learning. Some embodiments relate to a computer architecture for labeling documents.

### BACKGROUND

Many unlabeled documents exist. Labeling those documents may facilitate processing, storing, and retrieving the documents. For instance, a medical professional may generate multiple documents during an encounter with a patient. These documents may be associated with labels for processing by a payer, such as a government entity, an insurance company, or the patient him/herself. The labels may correspond to codes in a medical coding classification system, such as the International Classification of Diseases (ICD) and the Current Procedural Terminology (CPT). Techniques for automatically associating labels (e.g. codes in the medical coding classification system) with documents (e.g. documents representing an encounter with a patient) may be desirable.

### BRIEF DESCRIPTION OF THE DRAWINGS

FIG. 1 illustrates the training and use of a machine-learning program, in accordance with some embodiments.

FIG. 2 illustrates an example neural network, in accordance with some embodiments.

FIG. 3 illustrates the feature-extraction process and classifier training, in accordance with some embodiments.

FIG. 4 is a block diagram of a computing machine, in accordance with some embodiments.

FIG. 5 is a data flow diagram of assigning codes in a medical coding system to documents from an encounter, in accordance with some embodiments.

FIG. 6 illustrates a machine learning model architecture for labeling documents, in accordance with some embodiments.

FIG. 7 illustrates an example system for labeling documents, in accordance with some embodiments.

FIG. 8 illustrates an example system for generating a document-label map, in accordance with some embodiments.

FIG. 9 is a flow chart of a method for training a combined document-label association module, in accordance with some embodiments.

FIG. 10 is a flow chart of a method for generating a document map, in accordance with some embodiments.

FIG. 11 is a flow chart of a method for labeling documents, in accordance with some embodiments.

### SUMMARY

According to some aspects of the technology described herein, a system comprises processing circuitry; and a memory storing instructions which, when executed by the processing circuitry, cause the processing circuitry to perform operations comprising: accessing a collection of documents corresponding to a medical encounter and a labeling for the collection, wherein the labeling comprises one or more labels representing medical annotations assigned to the medical encounter; computing, using a Hierarchical Attention Network (HAN), for each of a plurality of document-label pairs, a probability that a document of the document-label pair corresponds to a label of the document-label pair based on one or more features of text in the document, wherein each document-label pair comprises a document from the collection of documents and a label from the labeling; and providing an output representing the computed probabilities.

According to some aspects of the technology described herein, a system comprises processing circuitry; and a memory storing instructions which, when executed by the processing circuitry, cause the processing circuitry to perform operations comprising: accessing a set of labels and a set of documents; assigning, to each label in the set of labels based on text associated with the label, one or more Natural Language Processing (NLP) content items; assigning, to each document in the set of documents based on text in the document, one or more NLP content items; mapping each document in at least a subset of the set of documents to one or more labels from the set of labels based on a correspondence between at least one NLP content item assigned to a given document from the subset and at least one NLP content item assigned to a given label from the set of labels to generate a document-label map; and providing an output representing at least a portion of the document-label map.

Other aspects include a method to perform the operations above, and a machine-readable medium storing instructions to perform the above operations.

### DETAILED DESCRIPTION

The following description and the drawings sufficiently illustrate specific embodiments to enable those skilled in the art to practice them. Other embodiments may incorporate structural, logical, electrical, process, and other changes. Portions and features of some embodiments may be included in, or substituted for, those of other embodiments. Embodiments set forth in the claims encompass all available equivalents of those claims.

As discussed above, techniques for automatically associating labels (e.g. codes in a medical coding classification system, such as the International Classification of Diseases (ICD) and the Current Procedural Terminology (CPT)) with documents (e.g. documents representing an encounter with a patient) may be desirable. Some aspects of the technology disclosed herein leverage machine learning to automatically associate labels with documents. Advantageously, in the medical coding classification context, document(s) associated with an encounter are automatically assigned the proper codes, ensuring efficient and accurate billing and payment processing, while saving person-hours in creating and reviewing billing records.

According to some aspects, a computing machine accesses a collection of documents corresponding to a medical encounter and a labeling for the collection. The labeling includes one or more (or zero or more) labels. The label(s) represent medical annotations (e.g. medical billing codes or medical concepts) assigned to the medical encounter. The computing machine computes, using a Hierarchical Attention Network (HAN), for each of a plurality of document-label pairs, a probability that a document of the document-label pair corresponds to a label of the document-label pair based on one or more features of text in the document. Each document-label pair includes a document from the collection of documents and a label from the labeling. The computing machine provides an output representing the computed probabilities.

In some cases, the HAN is trained using a document-label map. The document-label map is generated by a computing device (which is the same as or different from the computing machine). The computing device accesses a set of training labels and a set of training documents. The computing device assigns, to each training label in the set of training labels based on text associated with the training label, one or more Natural Language Processing (NLP) content items. The computing device assigns, to each training document in the set of training documents based on text in the training document, one or more NLP content items. The computing device maps each training document in at least a subset of the set of training documents to one or more training labels from the set of training labels based on a correspondence between at least one NLP content item assigned to a given training document from the subset and at least one NLP content item assigned to a given training label from the set of training labels to generate the document-label map.

In some cases, generating the document-label map also includes adding, to the document-label map a human-generated document-label association.

In some cases, the output representing the computed probabilities (generated by the computing machine) includes a collection of document-label pairs for which the probability exceeds a predetermined threshold. The output is provided to a user for verification that each document-label pair in the collection is correct. The computing device further trains the HAN based on the verification by the user.

According to some aspects, a first computer accesses an ordered set of labels and a training set of documents. At least a portion of the documents in the training set of documents are labeled with one or more labels from the ordered set of labels. The set of labels is ordered by number of documents to which each label is assigned, with the labels having the largest number of documents occurring first. The first computer trains, using the training set of documents, a first document-label association module to identify documents associated with a first label from the ordered set of labels. This training could be accomplished, for example, using supervised learning. The first computer trains, using the training set of documents, a second document-label association module to identify documents associated with a second label from the ordered set of labels. The second document-label association module is initialized based on the trained first document-label association module. The first computer provides, as a digital transmission (e.g. to a second computer different from the first computer), a representation of a combined document-label association module, which includes the first document-label association module and the second document-label association module.

In some cases, the first computer trains, using the training set of documents, a third document-label association module to identify documents associated with a third label from the ordered set of labels. The third document-label association module is initialized based on one or more of the trained first document-label association module and the trained second document-label association module. The combined document-label association module further includes the third document-label association module. Similar operations can occur with a fourth document-label association module for a fourth label, a fifth document-label association module for a fifth label, etc.

In some cases, the combined document-label association module is provided to a second computer different from the first computer. (Alternatively, the second computer and the first computer may be the same machine.) The second computer accesses a working set of documents. By executing the combined document-label association module, the second computer identifies an association between at least one document from the working set of documents and at least one label from the ordered set of labels.

As used herein, the phrases “computing machine,” “computing device,” and “computer” encompass their plain and ordinary meaning. These phrases may refer to, among other things, any single machine or combination of machines that includes processing circuitry and memory. These phrases may include one or more of a server, a client device, a desktop computer, a laptop computer, a mobile phone, a tablet computer, a personal digital assistant (PDA), a smart television, a smart watch, and the like.

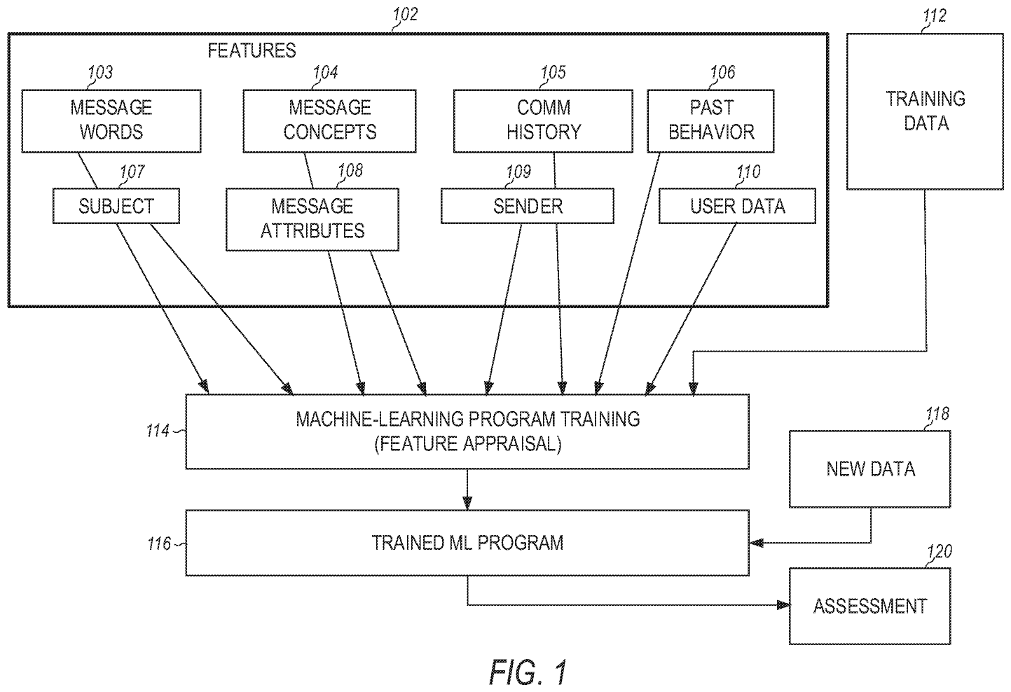

FIG. 1 illustrates the training and use of a machine-learning program, according to some example embodiments. In some example embodiments, machine-learning programs (MLPs), also referred to as machine-learning algorithms or tools, are utilized to perform operations associated with machine learning tasks, such as image recognition or machine translation.

Machine learning is a field of study that gives computers the ability to learn without being explicitly programmed. Machine learning explores the study and construction of algorithms, also referred to herein as tools, which may learn from existing data and make predictions about new data. Such machine-learning tools operate by building a model from example training data 112 in order to make data-driven predictions or decisions expressed as outputs or assessments 120. Although example embodiments are presented with respect to a few machine-learning tools, the principles presented herein may be applied to other machine-learning tools.

In some example embodiments, different machine-learning tools may be used. For example, Logistic Regression (LR), Naive-Bayes, Random Forest (RF), neural networks (NN), matrix factorization, and Support Vector Machines (SVM) tools may be used for classifying or scoring job postings.

Two common types of problems in machine learning are classification problems and regression problems. Classification problems, also referred to as categorization problems, aim at classifying items into one of several category values (for example, is this object an apple or an orange). Regression algorithms aim at quantifying some items (for example, by providing a value that is a real number). The machine-learning algorithms utilize the training data 112 to find correlations among identified features 102 that affect the outcome.

The machine-learning algorithms utilize features 102 for analyzing the data to generate assessments 120. A feature 102 is an individual measurable property of a phenomenon being observed. The concept of a feature is related to that of an explanatory variable used in statistical techniques such as linear regression. Choosing informative, discriminating, and independent features is important for effective operation of the MLP in pattern recognition, classification, and regression. Features may be of different types, such as numeric features, strings, and graphs.

In one example embodiment, the features 102 may be of different types and may include one or more of words of the message 103, message concepts 104, communication history 105, past user behavior 106, subject of the message 107, other message attributes 108, sender 109, and user data 110.

The machine-learning algorithms utilize the training data 112 to find correlations among the identified features 102 that affect the outcome or assessment 120. In some example embodiments, the training data 112 includes labeled data, which is known data for one or more identified features 102 and one or more outcomes, such as detecting communication patterns, detecting the meaning of the message, generating a summary of the message, detecting action items in the message, detecting urgency in the message, detecting a relationship of the user to the sender, calculating score attributes, calculating message scores, etc.

With the training data 112 and the identified features 102, the machine-learning tool is trained at operation 114. The machine-learning tool appraises the value of the features 102 as they correlate to the training data 112. The result of the training is the trained machine-learning program 116.

When the machine-learning program 116 is used to perform an assessment, new data 118 is provided as an input to the trained machine-learning program 116, and the machine-learning program 116 generates the assessment 120 as output. For example, when a message is checked for an action item, the machine-learning program utilizes the message content and message metadata to determine if there is a request for an action in the message.

Machine learning techniques train models to accurately make predictions on data fed into the models (e.g., what was said by a user in a given utterance; whether a noun is a person, place, or thing; what the weather will be like tomorrow). During a learning phase, the models are developed against a training dataset of inputs to optimize the models to correctly predict the output for a given input. Generally, the learning phase may be supervised, semi-supervised, or unsupervised; indicating a decreasing level to which the “correct” outputs are provided in correspondence to the training inputs. In a supervised learning phase, all of the outputs are provided to the model and the model is directed to develop a general rule or algorithm that maps the input to the output. In contrast, in an unsupervised learning phase, the desired output is not provided for the inputs so that the model may develop its own rules to discover relationships within the training dataset. In a semi-supervised learning phase, an incompletely labeled training set is provided, with some of the outputs known and some unknown for the training dataset.

Models may be run against a training dataset for several epochs (e.g., iterations), in which the training dataset is repeatedly fed into the model to refine its results. For example, in a supervised learning phase, a model is developed to predict the output for a given set of inputs, and is evaluated over several epochs to more reliably provide the output that is specified as corresponding to the given input for the greatest number of inputs for the training dataset. In another example, for an unsupervised learning phase, a model is developed to cluster the dataset into n groups, and is evaluated over several epochs as to how consistently it places a given input into a given group and how reliably it produces the n desired clusters across each epoch.

Once an epoch is run, the models are evaluated and the values of their variables are adjusted to attempt to better refine the model in an iterative fashion. In various aspects, the evaluations are biased against false negatives, biased against false positives, or evenly biased with respect to the overall accuracy of the model. The values may be adjusted in several ways depending on the machine learning technique used. For example, in a genetic or evolutionary algorithm, the values for the models that are most successful in predicting the desired outputs are used to develop values for models to use during the subsequent epoch, which may include random variation/mutation to provide additional data points. One of ordinary skill in the art will be familiar with several other machine learning algorithms that may be applied with the present disclosure, including linear regression, random forests, decision tree learning, neural networks, deep neural networks, etc.

Each model develops a rule or algorithm over several epochs by varying the values of one or more variables affecting the inputs to more closely map to a desired result, but as the training dataset may be varied, and is preferably very large, perfect accuracy and precision may not be achievable. A number of epochs that make up a learning phase, therefore, may be set as a given number of trials or a fixed time/computing budget, or may be terminated before that number/budget is reached when the accuracy of a given model is high enough or low enough or an accuracy plateau has been reached. For example, if the training phase is designed to run n epochs and produce a model with at least 95% accuracy, and such a model is produced before the n^{th} epoch, the learning phase may end early and use the produced model satisfying the end-goal accuracy threshold. Similarly, if a given model is inaccurate enough to satisfy a random chance threshold (e.g., the model is only 55% accurate in determining true/false outputs for given inputs), the learning phase for that model may be terminated early, although other models in the learning phase may continue training. Similarly, when a given model continues to provide similar accuracy or vacillate in its results across multiple epochs—having reached a performance plateau—the learning phase for the given model may terminate before the epoch number/computing budget is reached.

Once the learning phase is complete, the models are finalized. In some example embodiments, models that are finalized are evaluated against testing criteria. In a first example, a testing dataset that includes known outputs for its inputs is fed into the finalized models to determine an accuracy of the model in handling data that is has not been trained on. In a second example, a false positive rate or false negative rate may be used to evaluate the models after finalization. In a third example, a delineation between data clusterings is used to select a model that produces the clearest bounds for its clusters of data.

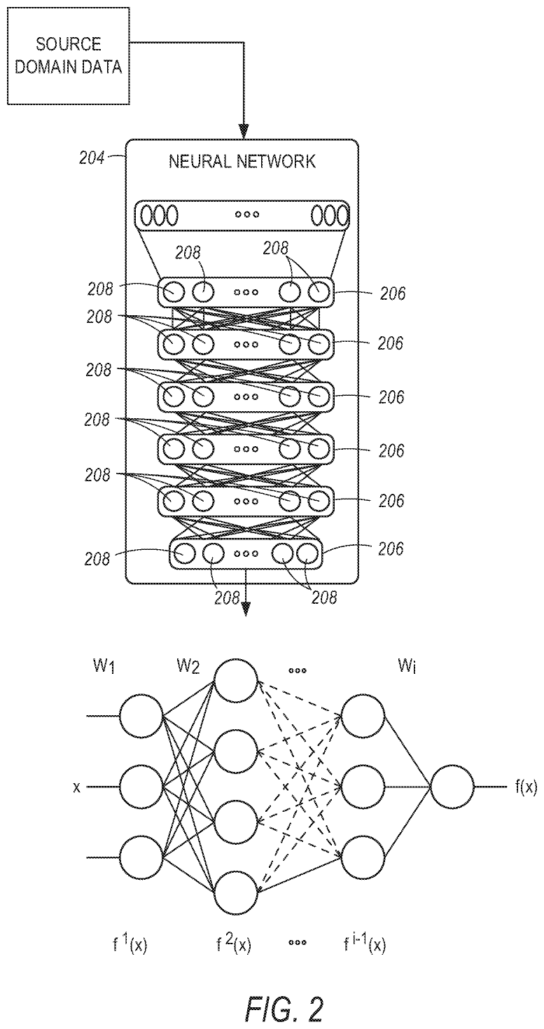

FIG. 2 illustrates an example neural network 204, in accordance with some embodiments. As shown, the neural network 204 receives, as input, source domain data 202. The input is passed through a plurality of layers 206 to arrive at an output. Each layer includes multiple neurons 208. The neurons 208 receive input from neurons of a previous layer and apply weights to the values received from those neurons in order to generate a neuron output. The neuron outputs from the final layer 206 are combined to generate the output of the neural network 204.

As illustrated at the bottom of FIG. 2, the input is a vector x. The input is passed through multiple layers 206, where weights W₁, W₂, . . . , Wᵢ are applied to the input to each layer to arrive at f¹(x), f²(x), . . . , fⁱ⁻¹(x), until finally the output f(x) is computed.

In some example embodiments, the neural network 204 (e.g., deep learning, deep convolutional, or recurrent neural network) includes a series of neurons 208, such as Long Short Term Memory (LSTM) nodes, arranged into a network. A neuron 208 is an architectural element used in data processing and artificial intelligence, particularly machine learning, which includes memory that may determine when to “remember” and when to “forget” values held in that memory based on the weights of inputs provided to the given neuron 208. Each of the neurons 208 used herein are configured to accept a predefined number of inputs from other neurons 208 in the neural network 204 to provide relational and sub-relational outputs for the content of the frames being analyzed. Individual neurons 208 may be chained together and/or organized into tree structures in various configurations of neural networks to provide interactions and relationship learning modeling for how each of the frames in an utterance are related to one another.

For example, an LSTM serving as a neuron includes several gates to handle input vectors (e.g., phonemes from an utterance), a memory cell, and an output vector (e.g., contextual representation). The input gate and output gate control the information flowing into and out of the memory cell, respectively, whereas forget gates optionally remove information from the memory cell based on the inputs from linked cells earlier in the neural network. Weights and bias vectors for the various gates are adjusted over the course of a training phase, and once the training phase is complete, those weights and biases are finalized for normal operation. One of skill in the art will appreciate that neurons and neural networks may be constructed programmatically (e.g., via software instructions) or via specialized hardware linking each neuron to form the neural network.

Neural networks utilize features for analyzing the data to generate assessments (e.g., recognize units of speech). A feature is an individual measurable property of a phenomenon being observed. The concept of feature is related to that of an explanatory variable used in statistical techniques such as linear regression. Further, deep features represent the output of nodes in hidden layers of the deep neural network.

A neural network, sometimes referred to as an artificial neural network, is a computing system/apparatus based on consideration of biological neural networks of animal brains. Such systems/apparatus progressively improve performance, which is referred to as learning, to perform tasks, typically without task-specific programming. For example, in image recognition, a neural network may be taught to identify images that contain an object by analyzing example images that have been tagged with a name for the object and, having learnt the object and name, may use the analytic results to identify the object in untagged images. A neural network is based on a collection of connected units called neurons, where each connection, called a synapse, between neurons can transmit a unidirectional signal with an activating strength that varies with the strength of the connection. The receiving neuron can activate and propagate a signal to downstream neurons connected to it, typically based on whether the combined incoming signals, which are from potentially many transmitting neurons, are of sufficient strength, where strength is a parameter.

A deep neural network (DNN) is a stacked neural network, which is composed of multiple layers. The layers are composed of nodes, which are locations where computation occurs, loosely patterned on a neuron in the human brain, which fires when it encounters sufficient stimuli. A node combines input from the data with a set of coefficients, or weights, that either amplify or dampen that input, which assigns significance to inputs for the task the algorithm is trying to learn. These input-weight products are summed, and the sum is passed through what is called a node's activation function, to determine whether and to what extent that signal progresses further through the network to affect the ultimate outcome. A DNN uses a cascade of many layers of non-linear processing units for feature extraction and transformation. Each successive layer uses the output from the previous layer as input. Higher-level features are derived from lower-level features to form a hierarchical representation. The layers following the input layer may be convolution layers that produce feature maps that are filtering results of the inputs and are used by the next convolution layer.

In training of a DNN architecture, a regression, which is structured as a set of statistical processes for estimating the relationships among variables, can include a minimization of a cost function. The cost function may be implemented as a function to return a number representing how well the neural network performed in mapping training examples to correct output. In training, if the cost function value is not within a pre-determined range, based on the known training images, backpropagation is used, where backpropagation is a common method of training artificial neural networks that are used with an optimization method such as a stochastic gradient descent (SGD) method.

Use of backpropagation can include propagation and weight update. When an input is presented to the neural network, it is propagated forward through the neural network, layer by layer, until it reaches the output layer. The output of the neural network is then compared to the desired output, using the cost function, and an error value is calculated for each of the nodes in the output layer. The error values are propagated backwards, starting from the output, until each node has an associated error value which roughly represents its contribution to the original output. Backpropagation can use these error values to calculate the gradient of the cost function with respect to the weights in the neural network. The calculated gradient is fed to the selected optimization method to update the weights to attempt to minimize the cost function.

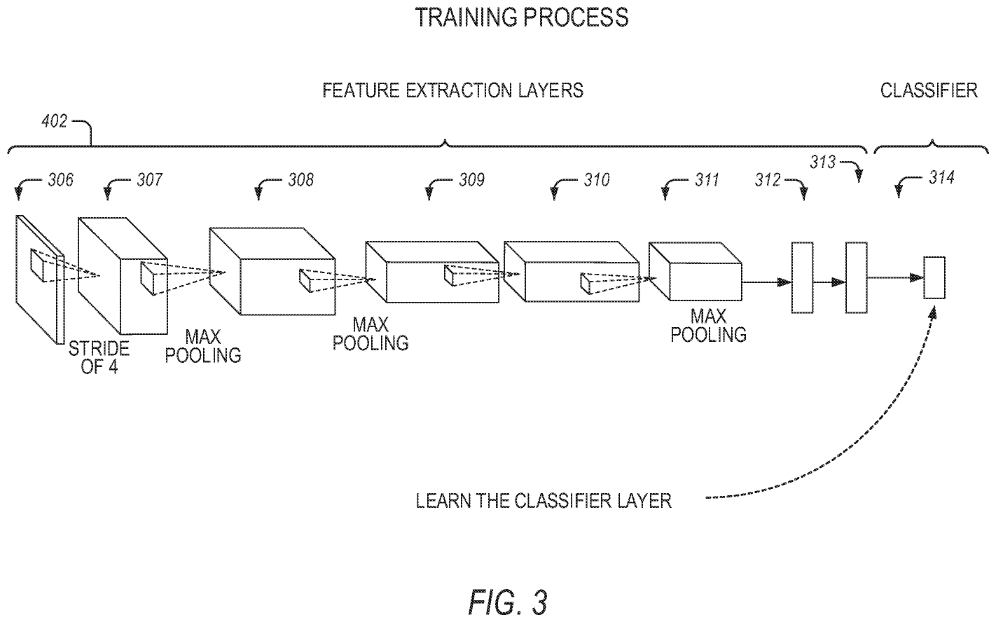

FIG. 3 illustrates the feature-extraction process and classifier training, according to some example embodiments. Training the classifier may be divided into feature extraction layers 302 and classifier layer 314. Each image is analyzed in sequence by a plurality of layers 306-313 in the feature-extraction layers 302.

Feature extraction is a process to reduce the amount of resources required to describe a large set of data. When performing analysis of complex data, one of the major problems stems from the number of variables involved. Analysis with a large number of variables generally requires a large amount of memory and computational power, and it may cause a classification algorithm to overfit to training samples and generalize poorly to new samples. Feature extraction is a general term describing methods of constructing combinations of variables to get around these large data-set problems while still describing the data with sufficient accuracy for the desired purpose.

In some example embodiments, feature extraction starts from an initial set of measured data and builds derived values (features) intended to be informative and non-redundant, facilitating the subsequent learning and generalization steps. Further, feature extraction is related to dimensionality reduction, such as be reducing large vectors (sometimes with very sparse data) to smaller vectors capturing the same, or similar, amount of information.

Determining a subset of the initial features is called feature selection. The selected features are expected to contain the relevant information from the input data, so that the desired task can be performed by using this reduced representation instead of the complete initial data. DNN utilizes a stack of layers, where each layer performs a function. For example, the layer could be a convolution, a non-linear transform, the calculation of an average, etc. Eventually this DNN produces outputs by classifier 314. In FIG. 3, the data travels from left to right and the features are extracted. The goal of training the neural network is to find the parameters of all the layers that make them adequate for the desired task.

As shown in FIG. 3, a “stride of 4” filter is applied at layer 306, and max pooling is applied at layers 307-313. The stride controls how the filter convolves around the input volume. “Stride of 4” refers to the filter convolving around the input volume four units at a time. Max pooling refers to down-sampling by selecting the maximum value in each max pooled region.

In some example embodiments, the structure of each layer is predefined. For example, a convolution layer may contain small convolution kernels and their respective convolution parameters, and a summation layer may calculate the sum, or the weighted sum, of two pixels of the input image. Training assists in defining the weight coefficients for the summation.

One way to improve the performance of DNNs is to identify newer structures for the feature-extraction layers, and another way is by improving the way the parameters are identified at the different layers for accomplishing a desired task. The challenge is that for a typical neural network, there may be millions of parameters to be optimized. Trying to optimize all these parameters from scratch may take hours, days, or even weeks, depending on the amount of computing resources available and the amount of data in the training set.

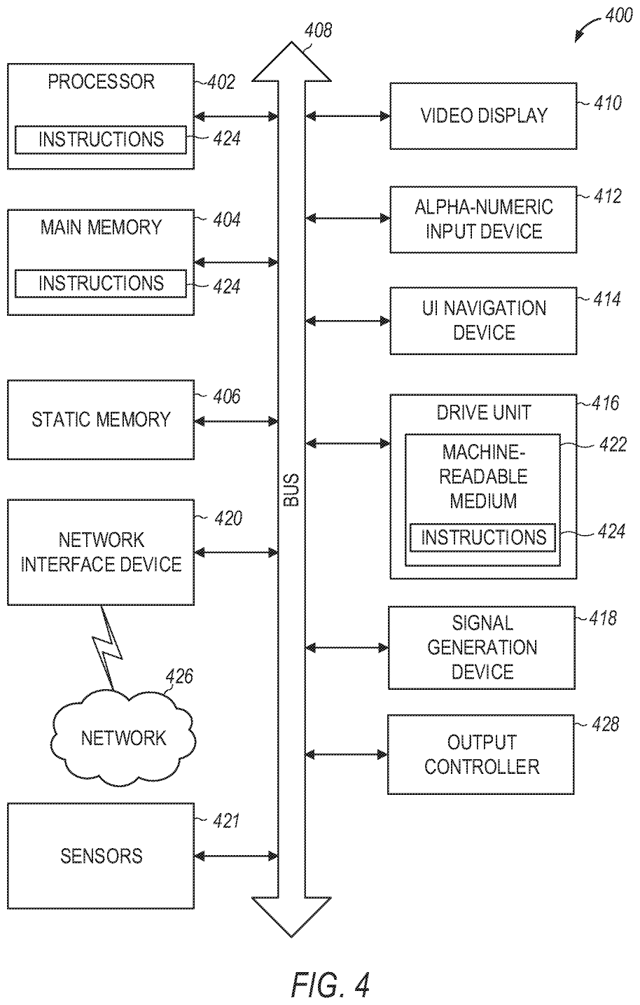

FIG. 4 illustrates a block diagram of a computing machine 400 in accordance with some embodiments. In some embodiments, the computing machine 400 may store the components shown in the circuit block diagram of FIG. 4. For example, the circuitry 400 may reside in the processor 402 and may be referred to as “processing circuitry.” Processing circuitry may include processing hardware, for example, one or more central processing units (CPUs), one or more graphics processing units (GPUs), and the like. In alternative embodiments, the computing machine 400 may operate as a standalone device or may be connected (e.g., networked) to other computers. In a networked deployment, the computing machine 400 may operate in the capacity of a server, a client, or both in server-client network environments. In an example, the computing machine 400 may act as a peer machine in peer-to-peer (P2P) (or other distributed) network environment. In this document, the phrases P2P, device-to-device (D2D) and sidelink may be used interchangeably. The computing machine 400 may be a specialized computer, a personal computer (PC), a tablet PC, a personal digital assistant (PDA), a mobile telephone, a smart phone, a web appliance, a network router, switch or bridge, or any machine capable of executing instructions (sequential or otherwise) that specify actions to be taken by that machine.

Examples, as described herein, may include, or may operate on, logic or a number of components, modules, or mechanisms. Modules and components are tangible entities (e.g., hardware) capable of performing specified operations and may be configured or arranged in a certain manner. In an example, circuits may be arranged (e.g., internally or with respect to external entities such as other circuits) in a specified manner as a module. In an example, the whole or part of one or more computer systems/apparatus (e.g., a standalone, client or server computer system) or one or more hardware processors may be configured by firmware or software (e.g., instructions, an application portion, or an application) as a module that operates to perform specified operations. In an example, the software may reside on a machine readable medium. In an example, the software, when executed by the underlying hardware of the module, causes the hardware to perform the specified operations.

Accordingly, the term “module” (and “component”) is understood to encompass a tangible entity, be that an entity that is physically constructed, specifically configured (e.g., hardwired), or temporarily (e.g., transitorily) configured (e.g., programmed) to operate in a specified manner or to perform part or all of any operation described herein. Considering examples in which modules are temporarily configured, each of the modules need not be instantiated at any one moment in time. For example, where the modules include a general-purpose hardware processor configured using software, the general-purpose hardware processor may be configured as respective different modules at different times. Software may accordingly configure a hardware processor, for example, to constitute a particular module at one instance of time and to constitute a different module at a different instance of time.

The computing machine 400 may include a hardware processor 402 (e.g., a central processing unit (CPU), a GPU, a hardware processor core, or any combination thereof), a main memory 404 and a static memory 406, some or all of which may communicate with each other via an interlink (e.g., bus) 408. Although not shown, the main memory 404 may contain any or all of removable storage and non-removable storage, volatile memory or non-volatile memory. The computing machine 400 may further include a video display unit 410 (or other display unit), an alphanumeric input device 412 (e.g., a keyboard), and a user interface (UI) navigation device 414 (e.g., a mouse). In an example, the display unit 410, input device 412 and UI navigation device 414 may be a touch screen display. The computing machine 400 may additionally include a storage device (e.g., drive unit) 416, a signal generation device 418 (e.g., a speaker), a network interface device 420, and one or more sensors 421, such as a global positioning system (GPS) sensor, compass, accelerometer, or other sensor. The computing machine 400 may include an output controller 428, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The drive unit 416 (e.g., a storage device) may include a machine readable medium 422 on which is stored one or more sets of data structures or instructions 424 (e.g., software) embodying or utilized by any one or more of the techniques or functions described herein. The instructions 424 may also reside, completely or at least partially, within the main memory 404, within static memory 406, or within the hardware processor 402 during execution thereof by the computing machine 400. In an example, one or any combination of the hardware processor 402, the main memory 404, the static memory 406, or the storage device 416 may constitute machine readable media.

While the machine readable medium 422 is illustrated as a single medium, the term “machine readable medium” may include a single medium or multiple media (e.g., a centralized or distributed database, and/or associated caches and servers) configured to store the one or more instructions 424.

The term “machine readable medium” may include any medium that is capable of storing, encoding, or carrying instructions for execution by the computing machine 400 and that cause the computing machine 400 to perform any one or more of the techniques of the present disclosure, or that is capable of storing, encoding or carrying data structures used by or associated with such instructions. Non-limiting machine readable medium examples may include solid-state memories, and optical and magnetic media. Specific examples of machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; Random Access Memory (RAM); and CD-ROM and DVD-ROM disks. In some examples, machine readable media may include non-transitory machine readable media. In some examples, machine readable media may include machine readable media that is not a transitory propagating signal.

The instructions 424 may further be transmitted or received over a communications network 426 using a transmission medium via the network interface device 420 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, a Long Term Evolution (LTE) family of standards, a Universal Mobile Telecommunications System (UMTS) family of standards, peer-to-peer (P2P) networks, among others. In an example, the network interface device 420 may include one or more physical jacks (e.g., Ethernet, coaxial, or phone jacks) or one or more antennas to connect to the communications network 426.

Some aspects of the technology described herein are directed to assigning labels (e.g., medical codes) to documents. One problem addressed by some aspects of the technology disclosed herein is that, in some cases, a NLP engine only receives individual document in a related set of documents (e.g. corresponding to a medial encounter) independently. In some implementations of NLP, no information about the related set of document is provided to the NLP engine. Thus, all the components of the NLP engine (e.g. machine learning components or rule-based annotators) are built on the document level, where documents are assigned codes.

Because NLP engine components only deal with individual document instead of the complete encounter together, it might be better to train/build the components with training data on document level (e.g. human codes on document). However, in most cases, the human codes are on the encounter level in the training data. Thus, some aspects assume that all the codes in the encounter level are also on each and every document that belongs to the encounter. This might mislead the training process, for example, forcing the pathology notes to produce radiology codes (or causing other incorrect codes to be produced).

In some aspects, a solution to the above problem uses attention network modeling. Some aspects train a model for each code to be able to assign a probability of the code on each document in the encounter. Based on the assigned probabilities, human codes on the encounter level may be assigned to specific document(s). With the document level code assignment, the NLP engine (using a machine learning coding model or using a rule-based approach) may be trained with document-specific coding information.

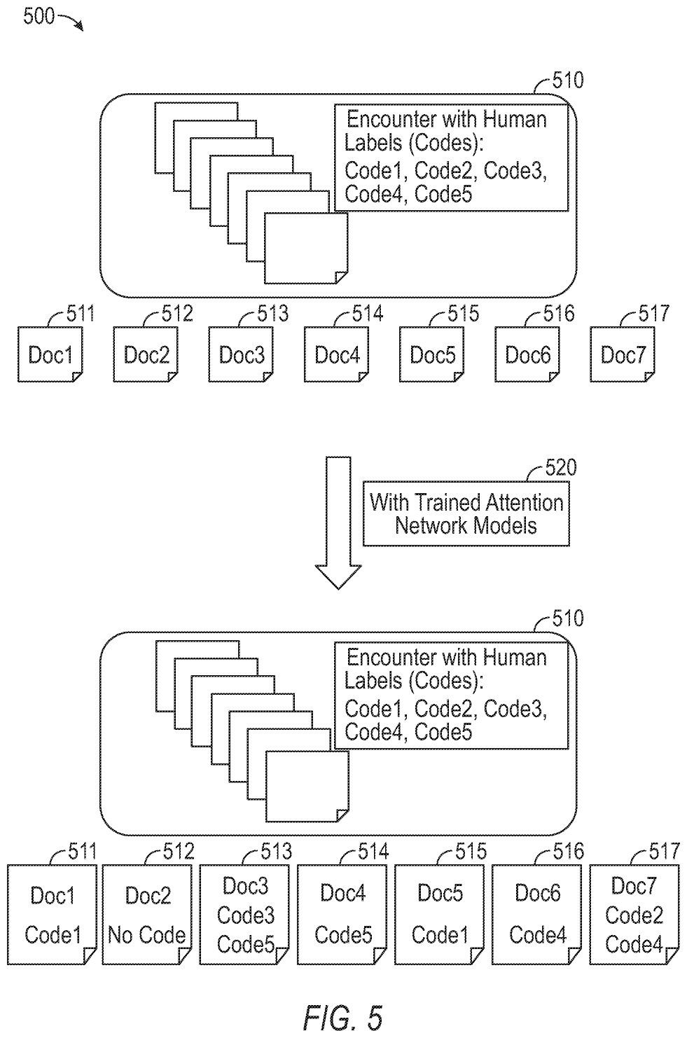

FIG. 5 is a data flow diagram 500 of assigning codes in a medical coding system to documents from an encounter, in accordance with some embodiments. As shown in the data flow diagram 500, an encounter 510 includes a set of documents and a set of codes/labels (Code1-Code5). The documents corresponds to documents (docs) 511-517. Trained attention network models 520 process the encounter 510 and its documents 511-517 to assign codes/labels to the documents. Each code/label is assigned to at least one document. Specifically, document 511 is associated with Code1. Document 512 is associated with no code/label. Document 513 is associated with Code3 and Code5. Document 514 is associated with Code5. Document 515 is associated with Code1. Document 516 is associated with Code4. Document 517 is associated with Code2 and Code4.

In some aspects, a training process includes the following operations: (1) collecting a training corpus with all the encounter human codes assigned to specific documents (either by human annotation, or through retrospective evidence filtering); (2) sorting codes based on occurrence time in the corpus from often to rare; (3) training attention network model for the most often code first; (4) continuing training the next less often code by transferring and initializing the network weights from the previous model training.

Applying the attention network models is done offline to prepare clean document level training data for medical coding model/rules. Given a collection of encounters, the attention models are applied to the encounters to assign encounter level codes to specific document(s).

The impacts to the NLP engine include the encounter codes being assigned to specific document(s). The NLP engine can be trained with clean data, and get improved performance. The NLP engine is the one deployed in production and process documents in the stream (e.g. not encounter). Some downstream process after NLP engine will rollup document level coding assigned by NLP engine into the encounter level.

Some aspects relate to a deep learning model that does encounter level coding by learning how much attention to pay to each of the documents in that encounter. If a document is more important in terms of helping the model to make a decision, it will receive more weight. The model jointly learns two things: (1) an encounter level medical code prediction model; and (2) an attention mechanism, i.e. a learnable smart weighted average of the documents in the encounter.

In the medical coding domain, data can be arranged in a hierarchical format. To be specific, a person can have several encounters, and an encounter can contain several documents. Traditionally, one may wish to build document level model. That is, one trains a model to predict what set of medical codes to assign to a document. However, in some cases, the ground truth of what set of medical codes each document contains is not available—only the ground truth of what set of medical codes each encounter contains is available. In other words, medical codes are stored at the encounter, not the document, level. Thus, there is a level mismatch problem between where the ground truth is (encounter) and where the data is (document).

One solution to the level mismatch problem is to train an encounter level model. One can either sum or average all the documents in the encounters to get an encounter representation and train a model with the encounter level ground truth label that exists. However, one downside of this approach is that signals of the codes could be average out or diluted when one is trying to average multiple documents.

Some aspects of the technology disclosed herein learn an attention model to do smarter averaging. Instead of treating all documents with the same weight, the model learns to assign more weights to documents that are more relevant in terms of predicting a code/label. It also jointly learns an encounter level model that take the weighted average documents and predict the encounter level codes.

With an encounter level attention neural network model, one can potentially add (or replace) an encounter level model into the production pipeline. With this model, one can accurately predict what codes to assign to the encounter, and which documents in that encounter is important in terms of that decision. This would lead to (1) a more accurate encounter model, and (2) a more interpretable (as one can see what documents are important) model.

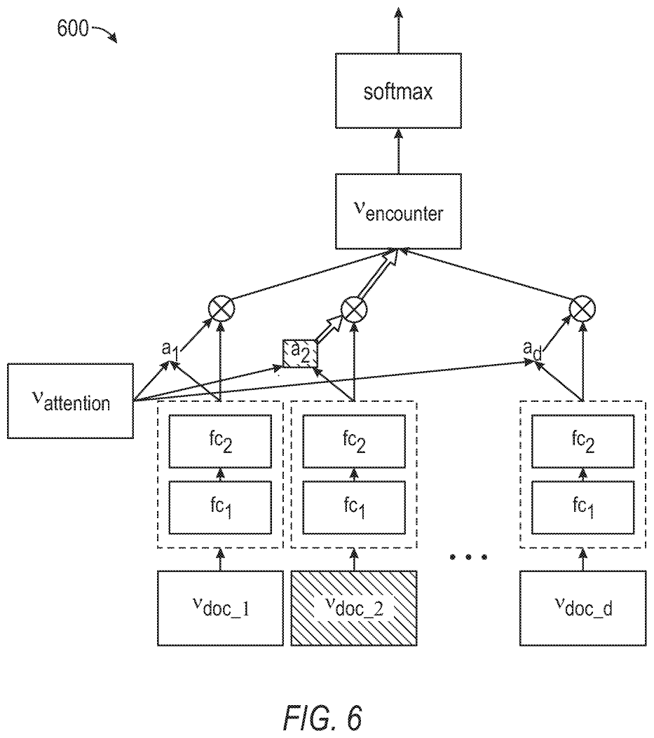

FIG. 6 illustrates a machine learning model architecture 600 for labeling documents, in accordance with some embodiments. In FIG. 6, v_{doc_d} refers to the vector representation of document d in the encounter, fc₁ and fc₂ are two fully connected layers (share among all documents). vₐₜₜₑₙₜᵢₒₙ is the attention mechanism that takes the output of the fully connected layer of a document and generate a weight (importance) a_{d} of the document. The model architecture 600 then computes the encounter level representation, v_{encounter}, by using a₁ . . . a_{d} as weights. Finally, the model architecture 600 passes v_{encounter} through a softmax layer to make a prediction on what medical codes to assign to the encounter. As shown, v_{doc_2} and a₂ demonstrate how weighted average can be done. If the model learns that v_{doc_2} is important, a₂ will be large, and thus the resulted weighted average (v_{encounter}) will appear closer to V_{doc_2}. In some cases, transfer learning can be used to initialize weights of the less frequent model with a trained more frequent model with the hope of reducing the amount of data needed.

As used herein, the term “softmax” encompasses its plain and ordinary meaning. In some examples, softmax is a function that takes as input a vector of K real numbers, and normalizes it into a probability distribution consisting of K probabilities. That is, prior to applying softmax, some vector components could be negative, or greater than one; and might not sum to 1; but after applying softmax, each component will be in the interval (0,1), and the components will add up to 1, so that they can be interpreted as probabilities. Furthermore, the larger input components will correspond to larger probabilities.

Attention neural network can be effective. However, it may not be optimal the targeted codes do not have enough data to train the model. Some aspects thus employ a naïve transfer learning approach by the following operations: (1) order the medical codes by frequency in terms of appearance in encounters; (2) train the model, m₀, with the most frequent medical code; (3) let mₚᵣₑᵥ←m₀; (4) train another model, mₙₑₓₜ, with the next frequent medical code by initializing the weight of mₙₑₓₜ with mₚᵣₑᵥ; (5) let mₚᵣₑᵥ←mₙₑₓₜ; (6) repeat operations 4-5 until all models are trained.

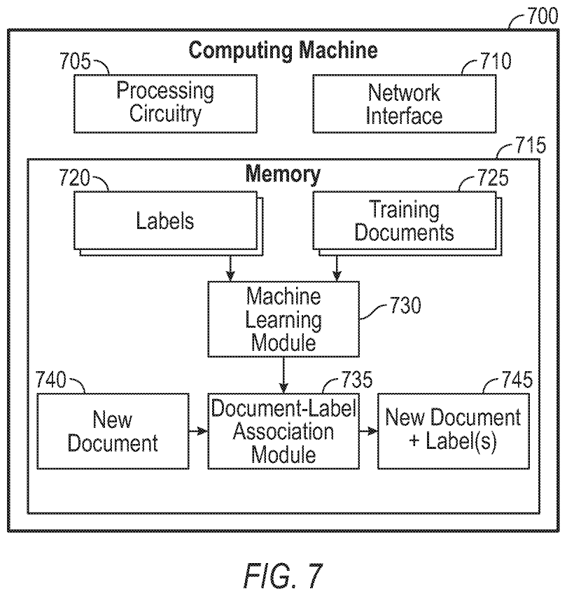

FIG. 7 illustrates an example system for labeling documents, in accordance with some embodiments. As shown, the system of FIG. 7 includes a computing machine 700, which includes processing circuitry 705, a network interface 710, and a memory 715. The processing circuitry 705 includes one or more processors and may be arranged into processing unit(s), such as CPU(s) or GPU(s). The network interface 710 includes one or more network interface cards (NICs), which cause the computing machine 700 to transmit and/or receive data in a network, such as the Internet. The network interface 710 may include one or more of a wired network interface, a wireless network interface, a local area network interface, a wide area network interface, and the like. The memory 715 stores data and/or instructions. As shown, the memory 715 includes labels 720, training documents 725, a machine learning module 730, a document-label association module 735, a new document 740, and a new document+label(s) combination 745.

In some aspects, the labels 720 correspond to medical codes. The labels 720 are ordered based on how many times they occur in the training documents 725 or another set of documents. At least a portion of the training documents 725 are labeled with one or more of the labels 720.

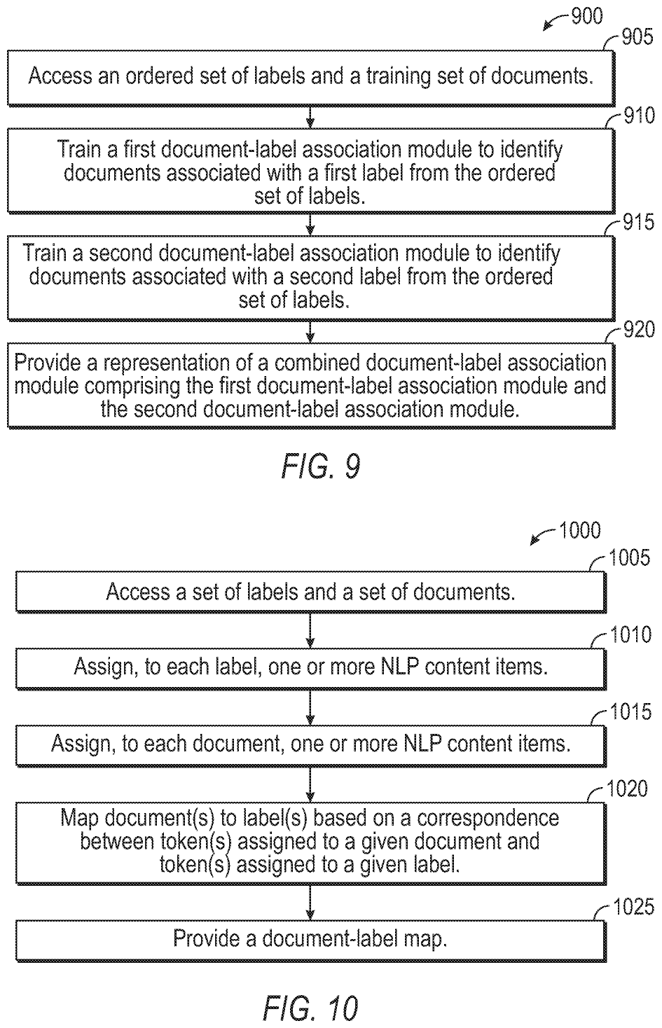

When executing the machine learning module 730, the processing circuitry 705 accesses the labels 720 and the training of documents 725. The processing circuitry 705 trains, using the training documents 725, the document-label association module 730 to identify documents associated with a first label from the ordered labels 720. The processing circuitry 705 trains, using the training documents 730, the document-label association module 735 to identify documents associated with a second label from the ordered labels 730. To identify the documents associated with the second label, the document-label association module is initialized based on weight(s) used to identify the documents associated with the first label. Similarly, the processing circuitry 705 trains the document-label association module 735 to identify documents associated with a third label, a fourth label, a fifth label, etc., from the ordered labels 720. The processing circuitry 705 provides an indication that the document-label association module 735 has been trained. In some cases, the trained document-label association module 735 may be transmitted to another machine for usage thereat. In some cases, a new document 740 is provided to the trained document-label association module 735. The processing circuitry 705, in executing the trained document-label association module 735, associates the new document 740 with one or more labels from the ordered label(s) 720. The processing circuitry 705 outputs a combination 745 of the new document and the one or more labels assigned to it (or an indication that no labels were assigned). More details of examples of the operation of the machine learning module 730 are provided in conjunction with FIG. 9.

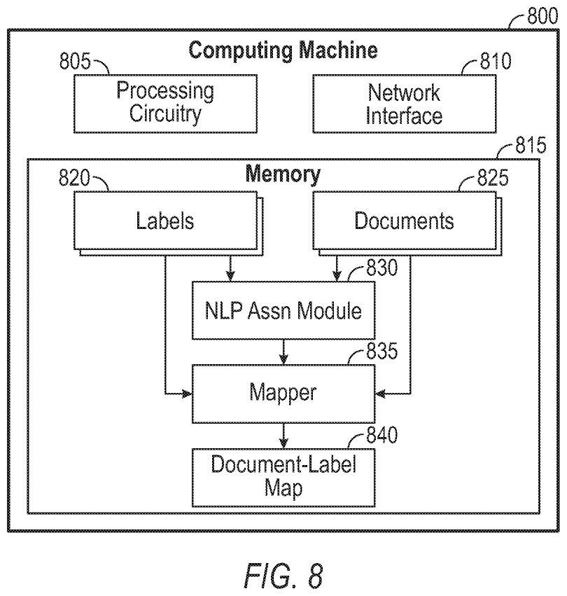

FIG. 8 illustrates an example system for generating a document-label map, in accordance with some embodiments. As shown, the system of FIG. 8 includes a computing machine 800, which includes processing circuitry 805, a network interface 810, and a memory 815. The processing circuitry 805 includes one or more processors and may be arranged into processing unit(s), such as CPU(s) or GPU(s). The network interface 810 includes one or more network interface cards (NICs), which cause the computing machine 800 to transmit and/or receive data in a network, such as the Internet. The network interface 810 may include one or more of a wired network interface, a wireless network interface, a local area network interface, a wide area network interface, and the like. The memory 815 stores labels 820, documents 825, a NLP association (assn) module 830, a mapper 835, and document-label map 840.

In some aspects, the labels 820 correspond to medical codes (e.g. of a given medical encounter). The documents 825 are associated with the labels (e.g., the documents are related to the given medical encounter).

The NLP association module 830 accesses the labels 820 and the documents 825. The NLP association module 830 assigns, to each label 820 based on the text associated with the label 820, one or more NLP content items from a set of NLP content items. The NLP association module 830 assigns, to each document 825 based on text in the document 825, one or more NLP content items from the set of NLP content items. The NLP content item(s) associated with each document 825 and each label 820 are provided to the mapper 835.

The mapper 835 maps each document in at least a subset of the documents 825 to one or more labels from the labels 820 based on a correspondence between at least one NLP content item assigned to a given document and at least one NLP content item assigned to a given label to generate the document-label map 840. In some cases, the given document is mapped to the given label if each and every NLP content item assigned to the given label is also assigned to the given document, and the given document is not mapped to the given label if there exists a NLP content item that is assigned to the given label and is not assigned. The mapper 835 outputs at least a portion of the document-label map 840.

FIG. 9 is a flow chart of a method 900 for training a combined document-label association module, in accordance with some embodiments. The method may be implemented by a computing machine (e.g. the computing machine 700 executing the machine learning module 730 and/or the document-label association module 735).

At operation 905, the computing machine accesses an ordered set of labels and a training set of documents. At least a portion of the documents in the training set of documents are labeled with one or more labels from the ordered set of labels. In some cases, the ordered set of labels is ordered based on a number of documents associated with each label in the training set of documents. In some cases, the ordered set of labels is ordered based on a number of documents associated with each label in a collection of documents different from the training set of documents. In some cases, the labels include codes from a medical coding classification system, and at least one document in the training set of documents is associated with a patient encounter.

At operation 910, the computing machine trains, using the training set of documents, a first document-label association module to identify documents associated with a first label from the ordered set of labels.

At operation 915, the computing machine trains, using the training set of documents, a second document-label association module to identify documents associated with a second label from the ordered set of labels. The second document-label association module is initialized based on the trained first document-label association module.

In some cases, the computing machine trains, using the training set of documents, a third document-label association module to identify documents associated with a third label from the ordered set of labels. The third document-label association module is initialized based on one or more of the trained first document-label association module and the trained second document-label association module. A fourth document-label association module for a fourth label, a fifth document-label association module for a fifth label, and the like may be similarly trained.

In some cases, the first document-label association module includes a first neural network with a plurality of first neurons. the plurality of first neurons are arranged in a plurality of first layers, the plurality of first layers including a first input layer, one or more first hidden layers, and a first output layer. In some cases, the second document-label association module includes a second neural network with a plurality of second neurons. The plurality of second neurons are arranged in a plurality of second layers, the plurality of second layers including a second input layer, one or more second hidden layers, and a second output layer, and where the plurality of second neurons are initialized based on the trained plurality of first neurons. The third, fourth, fifth, etc. document-label association modules may be structured similarly.

At operation 920, the computing machine provides, as a digital transmission, a representation of a combined document-label association module. The combined document-label association module includes at least the first document-label association module and the second document-label association module. The combined document-label association module may include each and every one of the first, second, third, fourth, fifth, etc., document-label association modules.

In some cases, the digital transmission is provided to a computing device different from the computing machine. (Alternatively, the computing device may be the same machine as the computing machine.) The computing device uses the combined document-label association module to access a working set of documents. The computing device uses the combined document-label association module to identify an association between at least one document from the working set of documents and at least one label from the ordered set of labels. After operation 920, the method 900 ends.

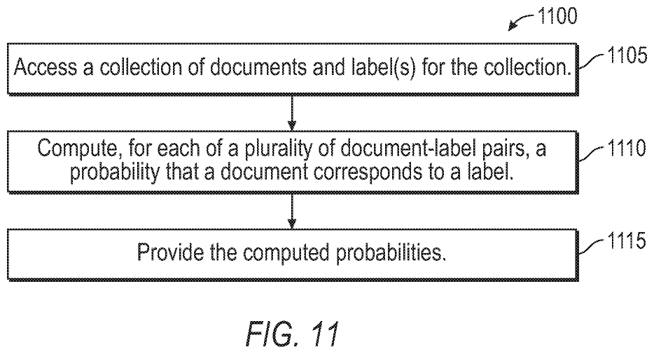

FIG. 10 is a flow chart of a method 1000 for generating a document map, in accordance with some embodiments. The method 1000 may be implemented by a computing machine (e.g. the computing machine 800 executing the NLP association module 830 and/or the mapper 835).

At operation 1005, the computing machine accesses a set of labels and a set of documents. In some cases, the set of labels includes codes from a medical coding classification system, and where the set of documents is associated with a patient encounter. The set of labels may include the medical code(s) assigned to that patient encounter.

At operation 1010, the computing machine assigns, to each label in the set of labels based on text associated with the label, one or more NLP content items.

At operation 1015, the computing machine assigns, to each document in the set of documents based on text in the document, one or more NLP content items.

At operation 1020, the computing machine maps each document in at least a subset of the set of documents to one or more labels from the set of labels based on a correspondence between at least one NLP content item assigned to a given document from the subset and at least one NLP content item assigned to a given label from the set of labels to generate a document-label map. In some cases, the given document is mapped to the given label if each and every NLP content item assigned to the given label is also assigned to the given document, and the given document is not mapped to the given label if there exists a NLP content item that is assigned to the given label and is not assigned to the given document.

At operation 1025, the computing machine provides an output representing the document-label map, for example, the document-label map itself. In some cases, the computing machine provides an output representing at least a portion of the document-label map. After operation 1025, the method 1000 ends.

In some cases, the document-label map generated by the method 1000 is used to train a HAN to compute a probability that a specified document corresponds to a specified label. In some cases, training the HAN to compute that probability includes the operations of: (1) ordering the labels in the set of labels based on a number of documents that correspond to each label to generate an ordered set of labels; (2) training, using the set of documents, a first document-label association module to identify documents associated with a first label from the ordered set of labels; (3) training, using the training set of documents, a second document-label association module to identify documents associated with a second label from the ordered set of labels, where the second document-label association module is initialized based on the trained first document-label association module; and (4) generating a combined document-label association module, where the combined document-label association module includes at least the first document-label association module and the second document-label association module. These operations correspond to those of the method 900 shown in FIG. 9. In some cases, the ordered set of labels orders the labels from largest corresponding number of documents to smallest corresponding number of documents. In some cases, training the HAN also includes: training, using the training set of documents, a third document-label association module to identify documents associated with a third label from the ordered set of labels, where the third document-label association module is initialized based on one or more of the trained first document-label association module and the trained second document-label association module, and where the combined document-label association module further includes the third document-label association module.

FIG. 11 is a flow chart of a method 1100 for labeling documents, in accordance with some embodiments. The method 1100 may be implemented by a computing machine.

At operation 1105, the computing machine accesses a collection of documents corresponding to a medical encounter and a labeling for the collection. The labeling includes one or more (or zero or more) labels representing medical annotations (e.g., medical billing codes or medical concepts) assigned to the medical encounter.

At operation 1110, the computing machine computes, using a HAN, for each of a plurality of document-label pairs, a probability that a document of the document-label pair corresponds to a label of the document-label pair based on one or more features of text in the document. Each document-label pair includes a document from the collection of documents and a label from the labeling. In some cases, if there are d documents in the collection and a labels in the labeling, there may be d*a document-label pairs in the plurality. Alternatively, only a subset of the d*a document-label pairs may be included in the plurality.

At operation 1115, the computing machine provides an output representing the computed probabilities. In some cases, the output representing the computed probabilities includes a collection of document-label pairs for which the probability exceeds a predetermined threshold. The output is provided to a user for verification that each document-label pair in the collection is correct.

The HAN may be further trained based on the verification by the user. After operation 1115, the method 1100 ends.

The HAN used in the method 1100 may be trained using a document-label map. The document-label map may be generated at a computer (that may be identical to or different from the computing machine) using operations including: (1) accessing a set of training labels and a set of training documents; (2) assigning, to each training label in the set of training labels based on text associated with the training label, one or more NLP content items; (3) assigning, to each training document in the set of training documents based on text in the training document, one or more NLP content items; and (4) mapping each training document in at least a subset of the set of training documents to one or more training labels from the set of training labels based on a correspondence between at least one NLP content item assigned to a given training document from the subset and at least one NLP content item assigned to a given training label from the set of training labels to generate the document-label map. These operations are similar to those of the method 1000 shown in FIG. 10. In some cases, generating the document-label map may also include: adding, to the document-label map a human-generated document-label association.

In some implementations described above, the collection of documents corresponds to a medical encounter, and the labeling represents medical annotations assigned to the medical encounter. However, the technology disclosed herein is not limited by these implementations. In alternative implementations, the collection of documents may correspond to any collection of documents and the labels may be any labels. In one example, the collection of documents corresponds to a legal document review project, and the labeling represents annotations assigned to the legal document review project. In one example, the collection of documents corresponds to a virtual book repository, and the labeling represents categories of books. In one example, the collection of documents corresponds to online posts, and the labeling represents tags of the online posts. Those skilled in the art may devise other things to which the collection of documents may correspond and/or other things which the labeling may represent.

Medical coding translates unstructured information about diagnoses, treatments, procedures, medications and equipment into alphanumeric codes, such as International Classification of Diseases (ICD) codes, or Current Procedural Terminology (CPT) codes, for billing or insurance purposes. To correctly interpret this information, experienced professionals (known as medical coders) are often involved in the process of medical coding. However, this can be expensive due to the large amount of medical text that needs to be processed and the high degree of expertise that is required.

A method that assists medical coders in automatic, or semi-automatic, assignment of medical codes can therefore be beneficial. Such a method should be able to suggest a set of possible codes for the coder to consider based on the information available in an encounter. A medical encounter may be to an interaction between a patient and healthcare provider, such as a patient visit to a hospital. This can range from a simple diagnoses report from a clinician, to a paper trail that may include admission diagnoses, radiology reports, progress and nursing notes, and discharge summary span over the duration of days or weeks. For simplicity, some aspects focus on the set of text documents generated during an encounter. Under this assumption, a medical encounter can be considered as an ordered collection of medical documents.

To efficiently assist coders, automatic medical code assignment might, in some cases, satisfy the following criteria: (1) high accuracy on code assignment, (2) the results are interpretable to the coder, and (3) the code is assigned at the encounter level, taking into account the collection of documents within the encounter.

Some aspects take into account that an encounter is a collection of documents, and directly train a model that predicts at the encounter level. This addresses the hierarchical structure of the medical encounter.

Medical coding may be treated as a classification problem. Document classification is an area in natural language processing. On the other hand, the classification of document collections, such as a medical encounter, presents a unique set of challenges.

One approach to address this problem is to train a document-level model: at prediction time, the predicted document codes are then merged into encounter codes. This approach has the benefit of reducing the problem to document classification. The challenge, however, is that when a medical coder assigns a medical code, it is on the medical encounter level. It is not always clear how to distribute the encounter-level code down to the document-level. The presence of a code in an encounter does not imply that all the documents in the encounter have evidence for that code. And information on specifically which documents are the “source” of the encounter-level code is also often not available from the medical coder. This inevitably leads to noise when training document-level model, as the ground-truth is meant to be assigned on the entire encounter. Merging document-level codes is also not a trivial task. In some cases, a more specific code may suppress more general codes, complicating the process.

The other approach to this problem is to train an encounter-level model directly. This approach has the benefit of not needing to worry about how document-level codes relate to the encounter-level. One naive way of training an encounter level model is to aggregate (either by summing or averaging) all the document features into a single encounter feature set. Doing that, however, might, in some cases, be noisy, as the signal of the targeted medical code could be diluted when irrelevant documents are also included. Another challenge is that the encounter-level result is to be interpretable by human coders, which calls for information about which documents of the medical encounter are the “source” of the medical code.

The Encounter-Level Document Attention Network (ELDAN) disclosed herein approaches these problems by operating at both the encounter and document level using attention. Attention enables the model to assign weights to different documents when combining them. This facilitates interpretation, in that it enables medical coder to interpret which documents are likely candidates for the code. This allows human to investigate specific documents, either to review the prediction, or to identify the problems of the prediction model.

Some contributions of some aspects of the technology disclosed herein include, among other things: (1) the application of hierarchical attention network to encounter-level coding, (2) implementation-level innovations needed to scale ELDAN up to a real-world number of codes, (3) evaluation not only of code quality, but of accuracy when identifying evidence for reviewers, and (4) transfer learning, which is effective for helping with rare codes.

The overall architecture of Encounter-Level Document Attention Network (ELDAN) is shown in FIG. 6, described above. It is a variant of a Hierarchical Attention Network (HAN) and includes three parts: (1) a document-level encoder that turns sparse document features into dense document features, (2) a document-level attention layer, and (3) an encounter-level encoder.

As multiple codes are often associated with an encounter, this can be considered as a multi-label classification problem. For simplicity, some aspects decompose the problem into multiple one-vs-all binary classification problem, with each one targeting a target code cₜ∈C={c₁, c₂, . . . , c_{K}}, the set of all codes. Let the set of encounters be E={e₁, e₂, . . . , eₙ}, and their corresponding labels be Y={y₁, y₂, . . . , yₙ}. Where yᵢ∈{−1,1} represents whether the encounter eᵢ contains the targeted medical code cᵣ. Each encounter eᵢ comprises multiple documents; the number of documents that an encounter contains can vary across encounters. Finally, let x_{i,j} and d_{i,j} be the sparse and dense feature vectors that represent document j in encounter i, respectively.

One goal of the document-level encoder is to transform a sparse document representation, x_{i,j}, into a dense document representation, d_{i,j}. The sparse document representation, x_{i,j} is first passed into an embedding layer, to map the sparse document representation into a vector. It is then followed by two fully connected layers to produce a dense document representation, d_{i,j}.

h_{i,j,0}=W_{Ebedding,x}_{_{i,j}} (1)

h_{i,j,1}=tan h(W_{FC}_{₁}h_{i,j,0}+b_{FC}_{₁}) (2)

d_{i,j}=tan h(W_{FC}_{₂}h_{i,j,1}+b_{FC}_{₂}) (3)

In the equations above, W represent weight matrix, b represent bias vector, and tan h is the hyperbolic tangent. h_{i,j,0} and h_{i,j,1} are hidden representations of the document j in encounter i.

When a medical code is assigned to an encounter, it does not imply all the documents that the encounter contains have evidence for the medical code. If the machine directly aggregates (whether by summing or averaging) all the dense document representations in that encounter, {d_{i,1}, d_{i,2}, . . . , d_{i,m}}, the machine might end up including irrelevant information that dilutes the signal of the presence of medical code. Instead, some aspects use a weighted average, where the more relevant documents are being paid more attention. To calculate attention for a document, the dense document representation d_{i,j} is compared to a learnable attention vector, vₐₜₜₑₙₜᵢₒₙ, after passing through a fully connected-layer and a non-linear layer. Specifically:

$$\begin{matrix}{u}_{i , j} = tan h ( {W}_{F {C}_{3}} {d}_{i , j} + {b}_{F {C}_{3}} ) & ( 4 ) \\ {a}_{i , j} = \frac{{u}_{i , j}^{T} {v}_{attention}}{{Σ}_{j = 1}^{m} {u}_{i , j}^{T} {v}_{attention}} & ( 5 ) \\ {e}_{i} {\sum }_{j = 1}^{m} {a}_{i , j} {d}_{i , j} & ( 6 )\end{matrix}$$

Above, a_{i,j} is the normalized attention score for document j in encounter i, and eᵢ is the encounter representation of encounter i. As shown in Equation 5, the transformed document representation u_{i,j} is compared with the learnable attention vector, v using dot product, and further normalized for the weighted averaging step in Equation 6.

Once the machine has the encounter representation eᵢ, the machine can predict whether the encounter contains the targeted medical code. Specifically:

P(ŷᵢ)=softmax(Wₑeᵢ+bₑ) (7)

Finally, the machine compares with the ground truth label of encounter i using negative log likelihood to calculate a loss on encounter i shown in Equation 8, where yᵢ is the ground-truth label.

Lossᵢ=−log(p(ŷᵢ=yᵢ)) (8)

Certain embodiments are described herein as numbered examples 1, 2, 3, etc. These numbered examples are provided as examples only and do not limit the technology disclosed herein.

Example 1 is a system comprising: processing circuitry; and a memory storing instructions which, when executed by the processing circuitry, cause the processing circuitry to perform operations comprising: accessing a collection of documents corresponding to a medical encounter and a labeling for the collection, wherein the labeling comprises one or more labels representing medical annotations assigned to the medical encounter; computing, using a Hierarchical Attention Network (HAN), for each of a plurality of document-label pairs, a probability that a document of the document-label pair corresponds to a label of the document-label pair based on one or more features of text in the document, wherein each document-label pair comprises a document from the collection of documents and a label from the labeling; and providing an output representing the computed probabilities.

In Example 2, the subject matter of Example 1 includes, wherein the medical annotations comprise medical billing codes or medical concepts.

In Example 3, the subject matter of Examples 1-2 includes, wherein the HAN is trained using a document-label map.

In Example 4, the subject matter of Example 3 includes, wherein the document-label map is generated by the processing circuitry performing operations comprising: accessing a set of training labels and a set of training documents; assigning, to each training label in the set of training labels based on text associated with the training label, one or more Natural Language Processing (NLP) content items; assigning, to each training document in the set of training documents based on text in the training document, one or more NLP content items; and mapping each training document in at least a subset of the set of training documents to one or more training labels from the set of training labels based on a correspondence between at least one NLP content item assigned to a given training document from the subset and at least one NLP content item assigned to a given training label from the set of training labels to generate the document-label map.

In Example 5, the subject matter of Example 4 includes, wherein the document-label map is generated by the processing circuitry further performing operations comprising: adding, to the document-label map a human-generated document-label association.

In Example 6, the subject matter of Examples 1-5 includes, wherein the output representing the computed probabilities comprises a collection of document-label pairs for which the probability exceeds a predetermined threshold, wherein the output is provided to a user for verification that each document-label pair in the collection is correct.

In Example 7, the subject matter of Example 6 includes, the operations further comprising: further training the HAN based on the verification by the user.

Example 8 is a system comprising: processing circuitry; and a memory storing instructions which, when executed by the processing circuitry, cause the processing circuitry to perform operations comprising: accessing a set of labels and a set of documents; assigning, to each label in the set of labels based on text associated with the label, one or more Natural Language Processing (NLP) content items; assigning, to each document in the set of documents based on text in the document, one or more NLP content items; mapping each document in at least a subset of the set of documents to one or more labels from the set of labels based on a correspondence between at least one NLP content item assigned to a given document from the subset and at least one NLP content item assigned to a given label from the set of labels to generate a document-label map; and providing an output representing at least a portion of the document-label map.

In Example 9, the subject matter of Example 8 includes, wherein the given document is mapped to the given label if each and every NLP content item assigned to the given label is also assigned to the given document, and wherein the given document is not mapped to the given label if there exists a NLP content item that is assigned to the given label and is not assigned to the given document.

In Example 10, the subject matter of Examples 8-9 includes, wherein the set of labels comprises codes from a medical coding classification system, and wherein the set of documents is associated with a patient encounter.

In Example 11, the subject matter of Example 10 includes, wherein the set of labels includes the codes that were assigned to the patient encounter.

In Example 12, the subject matter of Examples 8-11 includes, the operations further comprising: training, using the document-label map, a Hierarchical Attention Network (HAN) to compute a probability that a specified document corresponds to a specified label.

In Example 13, the subject matter of Example 12 includes, wherein training the HAN to compute the probability that the specified document corresponds to the specified label comprises: ordering the labels in the set of labels based on a number of documents that correspond to each label to generate an ordered set of labels; training, using the set of documents, a first document-label association module to identify documents associated with a first label from the ordered set of labels; training, using the training set of documents, a second document-label association module to identify documents associated with a second label from the ordered set of labels, wherein the second document-label association module is initialized based on the trained first document-label association module; and generating a combined document-label association module, wherein the combined document-label association module comprises at least the first document-label association module and the second document-label association module.

In Example 14, the subject matter of Example 13 includes, wherein the ordered set of labels orders the labels from largest corresponding number of documents to smallest corresponding number of documents.

In Example 15, the subject matter of Examples 13-14 includes, wherein training the HAN further comprises: training, using the training set of documents, a third document-label association module to identify documents associated with a third label from the ordered set of labels, wherein the third document-label association module is initialized based on one or more of the trained first document-label association module and the trained second document-label association module, and wherein the combined document-label association module further comprises the third document-label association module.

Example 16 is a machine-readable medium storing instructions which, when executed by processing circuitry of one or more machines, cause the processing circuitry to perform operations comprising: accessing a collection of documents corresponding to a medical encounter and a labeling for the collection, wherein the labeling comprises one or more labels representing medical annotations assigned to the medical encounter; computing, using a Hierarchical Attention Network (HAN), for each of a plurality of document-label pairs, a probability that a document of the document-label pair corresponds to a label of the document-label pair based on one or more features of text in the document, wherein each document-label pair comprises a document from the collection of documents and a label from the labeling; and providing an output representing the computed probabilities.

In Example 17, the subject matter of Example 16 includes, wherein the medical annotations comprise medical billing codes or medical concepts.

In Example 18, the subject matter of Examples 16-17 includes, wherein the HAN is trained using a document-label map.

In Example 19, the subject matter of Example 18 includes, wherein the document-label map is generated by the processing circuitry performing operations comprising: accessing a set of training labels and a set of training documents; assigning, to each training label in the set of training labels based on text associated with the training label, one or more Natural Language Processing (NLP) content items; assigning, to each training document in the set of training documents based on text in the training document, one or more NLP content items; and mapping each training document in at least a subset of the set of training documents to one or more training labels from the set of training labels based on a correspondence between at least one NLP content item assigned to a given training document from the subset and at least one NLP content item assigned to a given training label from the set of training labels to generate the document-label map.

In Example 20, the subject matter of Example 19 includes, wherein the document-label map is generated by the processing circuitry further performing operations comprising: adding, to the document-label map a human-generated document-label association.

In Example 21, the subject matter of Examples 16-20 includes, wherein the output representing the computed probabilities comprises a collection of document-label pairs for which the probability exceeds a predetermined threshold, wherein the output is provided to a user for verification that each document-label pair in the collection is correct.

In Example 22, the subject matter of Example 21 includes, the operations further comprising: further training the HAN based on the verification by the user.

Example 23 is a machine-readable medium storing instructions which, when executed by processing circuitry of one or more machines, cause the processing circuitry to perform operations comprising: accessing a set of labels and a set of documents; assigning, to each label in the set of labels based on text associated with the label, one or more Natural Language Processing (NLP) content items; assigning, to each document in the set of documents based on text in the document, one or more NLP content items; mapping each document in at least a subset of the set of documents to one or more labels from the set of labels based on a correspondence between at least one NLP content item assigned to a given document from the subset and at least one NLP content item assigned to a given label from the set of labels to generate a document-label map; and providing an output representing at least a portion of the document-label map.

In Example 24, the subject matter of Example 23 includes, wherein the given document is mapped to the given label if each and every NLP content item assigned to the given label is also assigned to the given document, and wherein the given document is not mapped to the given label if there exists a NLP content item that is assigned to the given label and is not assigned to the given document.

In Example 25, the subject matter of Examples 23-24 includes, wherein the set of labels comprises codes from a medical coding classification system, and wherein the set of documents is associated with a patient encounter.

In Example 26, the subject matter of Example 25 includes, wherein the set of labels includes the codes that were assigned to the patient encounter.

In Example 27, the subject matter of Examples 23-26 includes, the operations further comprising: training, using the document-label map, a Hierarchical Attention Network (HAN) to compute a probability that a specified document corresponds to a specified label.

In Example 28, the subject matter of Example 27 includes, wherein training the HAN to compute the probability that the specified document corresponds to the specified label comprises:

ordering the labels in the set of labels based on a number of documents that correspond to each label to generate an ordered set of labels; training, using the set of documents, a first document-label association module to identify documents associated with a first label from the ordered set of labels; training, using the training set of documents, a second document-label association module to identify documents associated with a second label from the ordered set of labels, wherein the second document-label association module is initialized based on the trained first document-label association module; and generating a combined document-label association module, wherein the combined document-label association module comprises at least the first document-label association module and the second document-label association module.

In Example 29, the subject matter of Example 28 includes, wherein the ordered set of labels orders the labels from largest corresponding number of documents to smallest corresponding number of documents.

In Example 30, the subject matter of Examples 28-29 includes, wherein training the HAN further comprises: training, using the training set of documents, a third document-label association module to identify documents associated with a third label from the ordered set of labels, wherein the third document-label association module is initialized based on one or more of the trained first document-label association module and the trained second document-label association module, and wherein the combined document-label association module further comprises the third document-label association module.

Example 31 is a method comprising: accessing a collection of documents corresponding to a medical encounter and a labeling for the collection, wherein the labeling comprises one or more labels representing medical annotations assigned to the medical encounter; computing, using a Hierarchical Attention Network (HAN), for each of a plurality of document-label pairs, a probability that a document of the document-label pair corresponds to a label of the document-label pair based on one or more features of text in the document, wherein each document-label pair comprises a document from the collection of documents and a label from the labeling; and providing an output representing the computed probabilities.

In Example 32, the subject matter of Example 31 includes, wherein the medical annotations comprise medical billing codes or medical concepts.

In Example 33, the subject matter of Examples 31-32 includes, wherein the HAN is trained using a document-label map.

In Example 34, the subject matter of Example 33 includes, wherein the document-label map is generated by the processing circuitry performing operations comprising: accessing a set of training labels and a set of training documents; assigning, to each training label in the set of training labels based on text associated with the training label, one or more Natural Language Processing (NLP) content items; assigning, to each training document in the set of training documents based on text in the training document, one or more NLP content items; and mapping each training document in at least a subset of the set of training documents to one or more training labels from the set of training labels based on a correspondence between at least one NLP content item assigned to a given training document from the subset and at least one NLP content item assigned to a given training label from the set of training labels to generate the document-label map.

In Example 35, the subject matter of Example 34 includes, wherein the document-label map is generated by the processing circuitry further performing operations comprising: adding, to the document-label map a human-generated document-label association.

In Example 36, the subject matter of Examples 31-35 includes, wherein the output representing the computed probabilities comprises a collection of document-label pairs for which the probability exceeds a predetermined threshold, wherein the output is provided to a user for verification that each document-label pair in the collection is correct.

In Example 37, the subject matter of Example 36 includes, the operations further comprising: further training the HAN based on the verification by the user.

Example 38 is a method comprising: accessing a set of labels and a set of documents; assigning, to each label in the set of labels based on text associated with the label, one or more Natural Language Processing (NLP) content items; assigning, to each document in the set of documents based on text in the document, one or more NLP content items; mapping each document in at least a subset of the set of documents to one or more labels from the set of labels based on a correspondence between at least one NLP content item assigned to a given document from the subset and at least one NLP content item assigned to a given label from the set of labels to generate a document-label map; and providing an output representing at least a portion of the document-label map.

In Example 39, the subject matter of Example 38 includes, wherein the given document is mapped to the given label if each and every NLP content item assigned to the given label is also assigned to the given document, and wherein the given document is not mapped to the given label if there exists a NLP content item that is assigned to the given label and is not assigned to the given document.

In Example 40, the subject matter of Examples 38-39 includes, wherein the set of labels comprises codes from a medical coding classification system, and wherein the set of documents is associated with a patient encounter.

In Example 41, the subject matter of Example 40 includes, wherein the set of labels includes the codes that were assigned to the patient encounter.

In Example 42, the subject matter of Examples 38-41 includes, the operations further comprising: training, using the document-label map, a Hierarchical Attention Network (HAN) to compute a probability that a specified document corresponds to a specified label.

In Example 43, the subject matter of Example 42 includes, wherein training the HAN to compute the probability that the specified document corresponds to the specified label comprises:

ordering the labels in the set of labels based on a number of documents that correspond to each label to generate an ordered set of labels; training, using the set of documents, a first document-label association module to identify documents associated with a first label from the ordered set of labels; training, using the training set of documents, a second document-label association module to identify documents associated with a second label from the ordered set of labels, wherein the second document-label association module is initialized based on the trained first document-label association module; and generating a combined document-label association module, wherein the combined document-label association module comprises at least the first document-label association module and the second document-label association module.

In Example 44, the subject matter of Example 43 includes, wherein the ordered set of labels orders the labels from largest corresponding number of documents to smallest corresponding number of documents.

In Example 45, the subject matter of Examples 43-44 includes, wherein training the HAN further comprises: training, using the training set of documents, a third document-label association module to identify documents associated with a third label from the ordered set of labels, wherein the third document-label association module is initialized based on one or more of the trained first document-label association module and the trained second document-label association module, and wherein the combined document-label association module further comprises the third document-label association module.

Example 46 is a system comprising: processing circuitry; and a memory storing instructions which, when executed by the processing circuitry, cause the processing circuitry to perform operations comprising: accessing a collection of documents and a labeling for the collection, wherein the labeling comprises one or more labels; computing, using a Hierarchical Attention Network (HAN), for each of a plurality of document-label pairs, a probability that a document of the document-label pair corresponds to a label of the document-label pair based on one or more features of text in the document, wherein each document-label pair comprises a document from the collection of documents and a label from the labeling; and providing an output representing the computed probabilities.

In Example 47, the subject matter of Example 46 includes, wherein the collection of documents corresponds to a medical encounter, and wherein the one or more labels represent medical annotations assigned to the medical encounter.

In Example 48, the subject matter of Examples 46-47 includes, wherein the collection of documents corresponds to a legal document review project, and wherein the one or more labels represent annotations assigned to the legal document review project.

In Example 49, the subject matter of Examples 46-48 includes, wherein the collection of documents corresponds to a virtual book repository, and wherein the one or more labels represent categories of books.

In Example 50, the subject matter of Examples 46-49 includes, wherein the collection of documents corresponds to online posts, and wherein the one or more labels represent tags of the online posts.

Example 51 is a machine-readable medium storing instructions which, when executed by processing circuitry of one or more machines, cause the processing circuitry to perform operations comprising: accessing a collection of documents and a labeling for the collection, wherein the labeling comprises one or more labels; computing, using a Hierarchical Attention Network (HAN), for each of a plurality of document-label pairs, a probability that a document of the document-label pair corresponds to a label of the document-label pair based on one or more features of text in the document, wherein each document-label pair comprises a document from the collection of documents and a label from the labeling; and providing an output representing the computed probabilities.

In Example 52, the subject matter of Example 51 includes, wherein the collection of documents corresponds to a medical encounter, and wherein the one or more labels represent medical annotations assigned to the medical encounter.

In Example 53, the subject matter of Examples 51-52 includes, wherein the collection of documents corresponds to a legal document review project, and wherein the one or more labels represent annotations assigned to the legal document review project.

In Example 54, the subject matter of Examples 51-53 includes, wherein the collection of documents corresponds to a virtual book repository, and wherein the one or more labels represent categories of books.

In Example 55, the subject matter of Examples 51-54 includes, wherein the collection of documents corresponds to online posts, and wherein the one or more labels represent tags of the online posts.

Example 56 is a method comprising: accessing a collection of documents and a labeling for the collection, wherein the labeling comprises one or more labels; computing, using a Hierarchical Attention Network (HAN), for each of a plurality of document-label pairs, a probability that a document of the document-label pair corresponds to a label of the document-label pair based on one or more features of text in the document, wherein each document-label pair comprises a document from the collection of documents and a label from the labeling; and providing an output representing the computed probabilities.

In Example 57, the subject matter of Example 56 includes, wherein the collection of documents corresponds to a medical encounter, and wherein the one or more labels represent medical annotations assigned to the medical encounter.

In Example 58, the subject matter of Examples 56-57 includes, wherein the collection of documents corresponds to a legal document review project, and wherein the one or more labels represent annotations assigned to the legal document review project.

In Example 59, the subject matter of Examples 56-58 includes, wherein the collection of documents corresponds to a virtual book repository, and wherein the one or more labels represent categories of books.

In Example 60, the subject matter of Examples 56-59 includes, wherein the collection of documents corresponds to online posts, and wherein the one or more labels represent tags of the online posts.

Example 61 is at least one machine-readable medium including instructions that, when executed by processing circuitry, cause the processing circuitry to perform operations to implement of any of Examples 1-60.

Example 62 is an apparatus comprising means to implement of any of Examples 1-60.

Example 63 is a system to implement of any of Examples 1-60.

Example 64 is a method to implement of any of Examples 1-60.

Although an embodiment has been described with reference to specific example embodiments, it will be evident that various modifications and changes may be made to these embodiments without departing from the broader spirit and scope of the present disclosure. Accordingly, the specification and drawings are to be regarded in an illustrative rather than a restrictive sense. The accompanying drawings that form a part hereof show, by way of illustration, and not of limitation, specific embodiments in which the subject matter may be practiced. The embodiments illustrated are described in sufficient detail to enable those skilled in the art to practice the teachings disclosed herein. Other embodiments may be utilized and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, user equipment (UE), article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract of the Disclosure is provided to comply with 37 C.F.R. § 1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment.

What is claimed is:

1. A system comprising:
processing circuitry; and
a memory storing instructions which, when executed by the processing circuitry, cause the processing circuitry to perform operations comprising:
accessing a collection of documents corresponding to a medical encounter and a labeling for the collection, wherein the labeling comprises one or more labels representing medical annotations assigned to the medical encounter;
computing, using a Hierarchical Attention Network (HAN), for each of a plurality of document-label pairs, a probability that a document of the document-label pair corresponds to a label of the document-label pair based on one or more features of text in the document, wherein each document-label pair comprises a document from the collection of documents and a label from the labeling; and
providing an output representing the computed probabilities.

2. The system of claim 1, wherein the medical annotations comprise medical billing codes or medical concepts.

3. The system of claim 1, wherein the HAN is trained using a document-label map.

4. The system of claim 3, wherein the document-label map is generated by the processing circuitry performing operations comprising:
accessing a set of training labels and a set of training documents;
assigning, to each training label in the set of training labels based on text associated with the training label, one or more Natural Language Processing (NLP) content items;
assigning, to each training document in the set of training documents based on text in the training document, one or more NLP content items; and
mapping each training document in at least a subset of the set of training documents to one or more training labels from the set of training labels based on a correspondence between at least one NLP content item assigned to a given training document from the subset and at least one NLP content item assigned to a given training label from the set of training labels to generate the document-label map.

5. The system of claim 4, wherein the document-label map is generated by the processing circuitry further performing operations comprising:
adding, to the document-label map a human-generated document-label association.

6. The system of claim 1, wherein the output representing the computed probabilities comprises a collection of document-label pairs for which the probability exceeds a predetermined threshold, wherein the output is provided to a user for verification that each document-label pair in the collection is correct.

7. The system of claim 6, the operations further comprising:
further training the HAN based on the verification by the user.

8. A system comprising:
processing circuitry; and
a memory storing instructions which, when executed by the processing circuitry, cause the processing circuitry to perform operations comprising:
accessing a set of labels and a set of documents;
assigning, to each label in the set of labels based on text associated with the label, one or more Natural Language Processing (NLP) content items;
assigning, to each document in the set of documents based on text in the document, one or more NLP content items;
mapping each document in at least a subset of the set of documents to one or more labels from the set of labels based on a correspondence between at least one NLP content item assigned to a given document from the subset and at least one NLP content item assigned to a given label from the set of labels to generate a document-label map; and
providing an output representing at least a portion of the document-label map.

9. The system of claim 8, wherein the given document is mapped to the given label if each and every NLP content item assigned to the given label is also assigned to the given document, and wherein the given document is not mapped to the given label if there exists a NLP content item that is assigned to the given label and is not assigned to the given document.

10. The system of claim 8, wherein the set of labels comprises codes from a medical coding classification system, and wherein the set of documents is associated with a patient encounter.

11. The system of claim 10, wherein the set of labels includes the codes that were assigned to the patient encounter.

12. The system of claim 8, the operations further comprising:
training, using the document-label map, a Hierarchical Attention Network (HAN) to compute a probability that a specified document corresponds to a specified label.

13. The system of claim 12, wherein training the HAN to compute the probability that the specified document corresponds to the specified label comprises:
ordering the labels in the set of labels based on a number of documents that correspond to each label to generate an ordered set of labels;
training, using the set of documents, a first document-label association module to identify documents associated with a first label from the ordered set of labels;
training, using the training set of documents, a second document-label association module to identify documents associated with a second label from the ordered set of labels, wherein the second document-label association module is initialized based on the trained first document-label association module; and
generating a combined document-label association module, wherein the combined document-label association module comprises at least the first document-label association module and the second document-label association module.

14. The system of claim 13, wherein the ordered set of labels orders the labels from largest corresponding number of documents to smallest corresponding number of documents.

15. The system of claim 13, wherein training the HAN further comprises:
training, using the training set of documents, a third document-label association module to identify documents associated with a third label from the ordered set of labels, wherein the third document-label association module is initialized based on one or more of the trained first document-label association module and the trained second document-label association module, and wherein the combined document-label association module further comprises the third document-label association module.

16. A method comprising:
accessing a collection of documents corresponding to a medical encounter and a labeling for the collection, wherein the labeling comprises one or more labels representing medical annotations assigned to the medical encounter;
computing, using a Hierarchical Attention Network (HAN), for each of a plurality of document-label pairs, a probability that a document of the document-label pair corresponds to a label of the document-label pair based on one or more features of text in the document, wherein each document-label pair comprises a document from the collection of documents and a label from the labeling; and providing an output representing the computed probabilities.

17. The method of claim 16, wherein the medical annotations comprise medical billing codes or medical concepts.

18. The method of claim 16, wherein the HAN is trained using a document-label map.

19. The method of claim 18, wherein the document-label map is generated by the processing circuitry performing operations comprising: accessing a set of training labels and a set of training documents;
assigning, to each training label in the set of training labels based on text associated with the training label, one or more Natural Language Processing (NLP) content items;
assigning, to each training document in the set of training documents based on text in the training document, one or more NLP content items; and mapping each training document in at least a subset of the set of training documents to one or more training labels from the set of training labels based on a correspondence between at least one NLP content item assigned to a given training document from the subset and at least one NLP content item assigned to a given training label from the set of training labels to generate the document-label map.

20. The method of claim 18, wherein the document-label map is generated by the processing circuitry further performing operations comprising: adding, to the document-label map a human-generated document-label association.
